(19) United States
(12) Patent Application Publication
(10) Pub. No.: US 2015/0376527 A1
(43) Pub. Date: Dec. 31, 2015

(54) CO2 CAPTURE FROM CO2-RICH NATURAL GAS

(71) Applicant: Jianguo XU, Wrightsown,, PA (US)

(72) Inventor: Jianguo XU, Wrightstown, PA (US)

(21) Appl. No.: 14/769,352

(22) PCT Filed: Feb. 21, 2014
(86) PCT No.: PCT/US14/17744

§ 371 Date: Aug. 20, 2015

Related U.S. Application Data
(60) Provisional application No. 61/767,791, filed on Feb. 21, 2013.
(60) Provisional application No. 61/818,885, filed on May 2, 2013.

(51) Int. Cl. C10L 3/10; C07C 7/00; F25J 3/02; C07C 7/08; C01B 31/20; B01D 53/14; C10G 7/08; C07C 7/11

(52) U.S. Cl. CPC C10L 3/104; C10G 7/08; C07C 7/005; C07C 7/11; C07C 7/08; C01B 31/20; B01D 53/1475; B01D 53/1493; F25J 3/0209; B01D 2252/2021

(57) ABSTRACT

It is described a prefractionation-physical absorption hybrid process and system for separation of a CO₂-natural gas mixture containing more than 26 mol % CO₂ and 0.1 mol % to 15 mol % in total one or more heavier hydrocarbons, such as ethane, propane, butanes, pentanes, and hexanes, with at least 0.0001 mol % in total one or more of pentanes and hexanes, particularly, a self-refrigerated process and system involving methanol as the absorbent.

### CROSS-REFERENCE TO RELATED APPLICATIONS

This application claims priority to U.S. Provisional Patent Application No. 61/767,791, filed on Feb. 21, 2013, and U.S. Provisional Patent Application No. 61/818,885, filed on May 2, 2013, each of which is hereby incorporated by reference herein in its entirety.

### BACKGROUND OF THE INVENTION

Some gases from oil wells or other sources contain both methane and high concentration levels, such as greater than 26 mol %, of carbon dioxide (CO₂). These gases normally contain smaller amounts of higher hydrocarbons, and sometimes also a small amount of nitrogen. It is desirable to separate CO₂ from methane to produce a methane gas with a CO₂ concentration typically of less than 2-4% so that it can be sold as a pipeline fuel gas while the captured CO₂ can be used for enhanced oil recovery or other purposes. This can be done by conventional CO₂ separation processes such as absorption with a high boiling solvent, such as dimethyl ethers of polyethylene glycols or a certain amine or amine mix, or adsorption by CO₂ selective adsorbents or membranes, or a combination thereof.

Processes using lower boiling CO₂ absorbents, e.g., physical solvents or absorbents, such as methanol, propylene carbonate, sulphinol, and N-Methyl-2-pyrrolidone (NMP), can absorb a significant amount of the higher hydrocarbons at the typical absorption conditions of the absorbers. However, due to the lower relative volatility of higher hydrocarbons with respect to the lower boiling CO₂ absorbents, it is difficult to separate the higher hydrocarbons from these absorbents. Therefore, processes using such lower boiling CO₂ absorbents are not desirable for separation of CO₂ from higher hydrocarbons in CO₂-containing gases. See for example, Burr and Lyddon, “A COMPARISON OF PHYSICAL SOLVENTS FOR ACID GAS REMOVAL”, 87th Gas Processors Association Annual Convention Proceedings 2008. This is particularly true for processes using methanol as the solvent, because the boiling points of some higher hydrocarbons are similar to or even higher than that of methanol, and separation by flashing or stripping of such higher hydrocarbons from the solvent is nearly impossible.

Hydrocarbons such as pentanes and hexanes can be used as an absorbent in the Ryan-Holmes process because there is no need for separation of the absorbent from the heavier product in such a process. However, energy consumption and capital cost are also very high in such a process.

SELEXOL™ (SELEXOL) process uses dimethyl ethers of polyethylene glycols as the solvent, whose boiling point is much higher than the other physical solvents. This process has been considered for removal of CO₂ and propane and heavier hydrocarbons from methane by Ross and Cuellar (F. Patrick Ross, P. E., Kyle T. Cuellar, “ECONOMICAL OPTION FOR CO₂/METHANE SEPARATION IN PRODUCED GAS CONTAINING A HIGH CO₂ FRACTION”, GasTech 2009). Ross and Cuellarller described the so called “Dual Refrigerant CO₂ Fractionation process” (or “the Ortloff DRCF process”) to first remove most of the CO₂ in the feed gas, along with the heavier components that are more difficult to be separated from the solvent before feeding the remaining gas mixture to the SELEXOL process. However, the power and equipment costs of the SELEXOL process are still very high.

The lower boiling physical absorbents, particularly methanol, have certain advantages over chemical absorbents and dimethyl ethers of polyethylene glycols. These advantages include, but are not limited to, lower viscosity, higher equilibrium CO₂ absorption capacity at the desired absorption temperature, lower heat capacity, and lower unit absorbent cost, etc. The lower boiling physical absorbents are suitable for working at lower temperatures so that the size of the absorption system can be reduced and corrosion issues in the absorption separation system essentially eliminated.

Thus, there is a need to develop more efficient process technology using a lower boiling physical absorbent for separation of CO₂ from a gas mixture containing CO₂, methane and higher hydrocarbons. Such need is met by the presently claimed invention.

### BRIEF SUMMARY OF THE INVENTION

It is surprisingly discovered in the present invention that CO₂ can be cost effectively separated from a gas mixture containing CO₂, methane and heavier hydrocarbons, using a distillation column (prefractionator) to first remove the heavier hydrocarbons to such a low level that a physical absorption process involving one or more lower boiling absorbents can also be used very efficiently. In addition, it is found that it is even possible to use a stripping section alone, i.e., at least one of the feed location is at the top of the distillation column, to remove the heavy hydrocarbons to levels acceptable for efficient operation of these physical absorption processes.

It is further discovered that such processes do not have to use external refrigerant for cooling. Instead, if the liquid CO₂-rich stream from the bottom of the prefractionator vaporizes at different pressures, and if one or more of the CO₂ vaporization pressures is below that of the prefractionator, the heat transfer ΔT's in the heat exchangers can be made more even, while the power needed to compress CO₂ to the desired pressure is greatly reduced, as is the capital cost of the compressors, resulting in great overall savings of capital and energy.

In a preferred embodiment of the present invention, even methanol, the physical solvent with the lowest boiling point among the non-hydrocarbon absorbents of interest, can be used as a solvent in a process or system of the present invention. Prefractionator with a stripping section can render the levels of hydrocarbons low enough for the methanol absorption process to work very efficiently according to embodiments of the present invention.

The present invention enables significant simplification of process systems and reduction of power consumption via elimination of external refrigeration source by splitting the CO₂-rich product (light components depleted product) stream from the bottom of the prefractionator into two or more substreams, setting their pressures to different levels, and using them to cool the feed mixture. Setting the pressure of the prefractionator at a different level from the pressures of the substreams of the light component-depleted product further reduces the compression duty of the system.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows an example of a CO₂-natural gas prefractionation separation process according to some embodiments of the present invention;

FIG. 2 shows an example of a CO₂-natural gas prefractionation-methanol absorption hybrid separation process according to some embodiments of the present invention.

### DETAILED DESCRIPTION OF THE INVENTION

Various publications and articles are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Embodiments of the present invention relate to a prefractionation-physical absorption hybrid process and system for separation of a CO₂-natural gas mixture containing more than 26 mol % CO₂ and 0.1 mol % to 15 mol % in total of heavier hydrocarbons, with at least 0.0001 mol % in total one or more of pentanes and hexanes, preferably in a self-refrigerated process.

As used herein, the term “heavier hydrocarbon” or “higher hydrocarbon” refers to any hydrocarbon having a carbon number of two or more that one may find in a natural gas. The “heavier hydrocarbon” can be a hydrocarbon having 2, 3, 4, 5, 6 or more carbon. Examples of “heavier hydrocarbon” include, but are not limited to, ethane, propane, butanes, pentanes, and hexanes, more particularly pentanes and hexanes.

As used herein, the term “light component” refers to a component within a gas mixture that has a boiling point lower than that of CO₂. Examples of the “light component” include methane and nitrogen.

Accordingly, in one general aspect, the present invention relates to a process for separating a stream of a feed mixture comprising greater than 26 mol % CO₂ and 0.1 mol % to 15 mol % in total of heavier hydrocarbons, wherein the feed mixture contains at least 0.0001 mol % in total one or more of pentanes and hexanes, using one or more physical solvents. The process comprises:
a) cooling at least a portion of the feed mixture in reboiler of a distillation column, feeding at least a portion of reboiler-cooled feed mixture to a second heat exchanger to further cool the feed mixture to below 32° F., and sending at least a portion of the feed mixture cooled to below 32° F. to the distillation column, wherein the feed mixture is separated into at least a first liquid stream containing 10 mol % or less light components and a second stream having a mole fraction of the light components higher than the mole fraction of the light components in the feed mixture;
b) producing the second stream from the top or near the top of the distillation column, and sending the second stream to a physical absorption unit using an absorbent comprising at least one selected from the group consisting of methanol, N-methyl-2-pyrrolidone, propylene carbonate, and sulphinol;
c) introducing the first stream from a position at or near the bottom of the distillation column to the reboiler, wherein the first stream is at least partially vaporized, and letting the at least partially vaporized first stream flow out of the reboiler;
d) routing at least a portion of the vapor of the at least partially vaporized first stream back to the distillation column, and producing a liquid product stream that is depleted with the light components.

In a preferred embodiment of the present invention, the feed mixture further comprises methane as the light component. For example, the feed mixture further comprises 2 mol % to 73.9 mol % methane.

As understood by those skilled in the art, a reboiler is a heat exchanger. It can have at least a stream to be heated and a stream to be cooled.

In an embodiment of a process of the present invention, the feed mixture is first cooled in a warmer section of the second heat exchanger before the feed mixture is fed to the reboiler, then a colder section of the second heat exchanger.

Obviously, if the pressure of the distillation column (prefractionator) is lower than that of the cooled feed mixture, a pressure reduction device such as a throttle valve is needed to reduce the pressure of the feed mixture before feeding this mixture to the prefractionator.

In an embodiment of a process of the present invention, the reboiler and the second heat exchanger are integrated into a combined heat exchanger.

Note the second heat exchanger or combined heat exchanger does not have to be a single physical heat exchanger. There can be two or more physical heat exchangers, in parallel or in series, or a combination of parallel and series arrangements. For example, the second heat exchanger can be split into two physical heat exchangers so that the feed mixture is first cooled in the warmer one, then cooled in the reboiler of the prefractionator, and then further cooled in the colder one.

The distillation column mentioned above is also called prefractionator to distinguish it from the other subsequent distillation column(s).

In an embodiment of a process of the present invention, the feed mixture cooled to below 32° F. is fed to the distillation column on or above the top stage or packing layer of the distillation column.

In another embodiment of a process of the present invention, at least a portion of the light components-depleted liquid product stream is heated in the second heat exchanger against cooling of the feed stream.

In another embodiment of a process of the present invention, at least a portion of the light components-depleted liquid product stream is sent to a second distillation column for further separation.

In yet another embodiment of a process of the present invention, the feed mixture cooled to below 32° F. is expanded in a dense fluid expander before being fed to the distillation column.

In yet another embodiment of a process of the present invention, the absorbent is substantially methanol.

In an embodiment of a process of the present invention, the feed mixture cooled to below 32° F. is expanded in a dense fluid expander before being fed to the distillation column.

In another general aspect, the present invention relates to a process for separating a stream of a feed mixture, wherein the refrigeration is solely provided by streams of the process, and wherein the feed mixture comprises greater than 26 mol % CO₂, and 0.1 mol % to 15 mol % in total in total of heavier hydrocarbons, wherein the feed mixture comprises at least 0.0001 mol % in total one or more of pentanes and hexanes. The process comprises:
a) cooling the feed mixture in reboiler of a distillation column, feeding the reboiler-cooled feed mixture to a second heat exchanger to further cool the feed mixture to below 32° F., and sending the resultant cooled feed mixture to a distillation column, wherein the feed mixture is separated into at least a first stream containing 10 mol % or less light components and a second stream having a mole fraction of the light components higher than the mole fraction of the light components of the feed mixture;
b) producing the second stream from the top or near the top of the distillation column, and sending the second stream to an absorption unit for further separation of CO₂ from methane;
c) introducing the first stream from a position at or near the bottom of the distillation column to the reboiler wherein the first stream is at least partially vaporized, routing the at least partially vaporized first stream out of the reboiler, and feeding at least a portion of the vapor of the resultant at least partially vaporized light components-depleted stream back to the distillation column;
d) producing a liquid product that is depleted with the light components;
e) splitting the liquid product that is depleted with the light components from the distillation column into at least two light components-depleted liquid product substreams,
f) letting down the pressure of a light components-depleted liquid product substream and sending it to a position in the colder section of the second heat exchanger wherein the light components-depleted liquid substream is heated, completely vaporized, and further heated as it travels from the colder section to the warmer section of the second heat exchanger, and taking the resultant vapor out of the second heat exchanger at or close to the warm end of the heat exchanger; and
g) boosting the pressure of a second light components-depleted liquid product substream to a level that is greater than that in the distillation column, sending the pressure-boosted second light components-depleted liquid product substream to an intermediate location of the second heat exchanger wherein the second light components-depleted liquid product substream is heated as it travels to the warmer section of the second heat exchanger, and taking the resultant heated stream out of the second heat exchanger at or close to its warm end.

In a preferred embodiment of the present invention, the feed mixture further comprises methane as the light component. For example, the feed mixture further comprises 2 mol % to 73.9 mol % methane.

Here “streams of the process” means the feed streams (including the feed mixture and makeup absorbent) and the streams produced from the feed streams. That is to say, “the refrigeration is solely provided by the streams of the process” means no external refrigeration is used. That reduces the complexity of the system by eliminating an external refrigeration loop that is needed in the Ortloff DRCF process, including the compressor(s), intercooler (if any), condenser, and throttle valve, and refrigerant of a reverse Rankine cycle refrigeration system (or compressor, aftercooler, expander, and refrigerant of a reverse Brayton cycle).

In an embodiment of the present invention, the feed mixture is first cooled in the warm section of the second heat exchanger before it is fed to the reboiler and the cold section of the second heat exchanger.

In an embodiment of the present invention, the reboiler and the second heat exchanger are integrated into a combined heat exchanger.

In another embodiment of the present invention, the absorption unit comprises an absorbent comprising one or more selected from the group consisting methanol, N-Methyl-2-pyrrolidone, propylene carbonate, and sulphinol.

In an embodiment of the present invention, the light components-depleted liquid product substream to be let down in pressure is first cooled to a temperature that is lower than the temperature at the bottom of the distillation column before its pressure is reduced.

In the third general aspect, the present invention relates to a system for separating a stream of a feed mixture, wherein the feed mixture comprises greater than 26 mol % CO₂, and 0.1 mol % to 15 mol % in total of heavier hydrocarbons, wherein the feed mixture comprises at least 0.0001 mol % in total one or more of pentanes and hexanes. The system comprises a distillation column with a reboiler, a second heat exchanger, and a physical absorption system having an absorbent comprising at least one of the components selected from the group consisting of methanol, N-Methyl-2-pyrrolidone, propylene carbonate, and sulphinol, the system further comprises:
a) a feed mixture inlet of the distillation column for receiving the feed mixture, a passage for the feed mixture to be cooled in the reboiler, a passage for cooling the feed mixture in the second heat exchanger, a conduit for connecting the reboiler with the feed mixture passage of the second heat exchanger, a conduit for connecting the feed mixture passage in the second heat exchanger with the feed mixture inlet of the distillation column, a first outlet on the distillation column, located at or near the bottom of the distillation column, for removing a first stream from the distillation column, a second outlet on the distillation column, located at or near the top of the distillation column, for removing said second stream from the distillation column, a conduit for connecting said second outlet on the distillation column with the physical absorption CO₂ removal unit, and
b) a passage in the reboiler for at least partially vaporizing said first stream, a means for connecting the distillation column at a position near its bottom with a first end of said passage of the reboiler, and a means for connecting a second end of said passage at the second end of the reboiler with the distillation column at a position near the bottom of the distillation column for admitting at least a portion of the vapor of the at least partially vaporized first stream from said passage in the reboiler into the distillation column, and an outlet for a liquid components-depleted liquid product stream to leave the distillation column.

In an embodiment of the system of the present invention, the second heat exchanger has a warmer section and a colder section, both warmer section and colder section having passages for cooling the feed mixture, a conduit for connecting the feed mixture passage in the warmer section of the second heat exchanger with the feed mixture passage in the reboiler, and a conduit for connecting the feed mixture passage in the reboiler with the feed mixture passage in the colder section of the second heat exchanger.

In an embodiment of the system of the present invention, the second heat exchanger and the reboiler are integrated into a combined heat exchanger.

In an embodiment of the system of the present invention, the feed mixture inlet of the distillation column is located on or above the top tray or layer of packing of the distillation column.

In another embodiment, the system of the present invention further comprises a passage in the second heat exchanger for heating at least a portion of the liquid product stream that is depleted with the light components and a means for connecting said passage for the light components-depleted liquid product stream with the distillation column.

In an embodiment, the system of the present invention, further comprises a second distillation column for further separation of at least a portion of the light components-depleted liquid product stream from the first distillation column and a means for connecting the first distillation column with the second distillation column.

In an embodiment of the system of the present invention, the absorbent of the CO₂ absorption unit in component b) is substantially methanol.

The first stream in step c) of the process according to embodiments of the present invention may or may not have the same composition as the light components-depleted liquid product stream in each of these general aspects. When the composition of the first stream is different from the light components-depleted liquid product stream, the first stream may be partially vaporized and sent to a phase separator, and the light components-depleted liquid product stream may be from this phase separator, and the vapor from the phase separator is sent back to the prefractionator. In other words, the light components depleted stream phase separator mentioned in this paragraph is considered a part, more specifically the bottom part, of the distillation in the context, including the claims, of this application.

Among the higher hydrocarbons contained in the feed gas, such as ethane, propane, iso-butane, n-butane, iso-pentanes, n-pentane, hexanes etc., ethane will likely be distributed between the first stream and the overhead light components enriched stream, as may some propane, while greater than 90 mol % of the butanes and essentially all (>99%) pentanes and higher hydrocarbons will go with the light components-depleted liquid.

If the feed pressure is high enough, under certain conditions, a portion of the light components-depleted liquid can be pumped to a pressure that is equal to or even higher than the pressure of the feed mixture, for example, to the desired CO₂ delivery pressure before it is heated in the second heat exchanger.

It is also preferable that the vaporization pressures of the light components depleted product substreams are different from the pressure in the bottom of the prefractionator.

The second heat exchanger or combined heat exchanger is likely a multi-passage plate-fin heat exchanger or several of such heat exchangers.

The second heat exchanger or combined heat exchanger can be orientated in such a way that the warm end is the lower end, and the cold end is the upper end. The light components-depleted liquid substream can be first sent to a phase separator after being letdown in pressure. The vapor and liquid streams can then be sent to the main heat exchanger in separate lines. Preferably, the phase separator is located at a position higher than the second or combined heat exchanger so that the liquid from the phase separator can flow into the heat exchanger without a pump. The pressure drops in the passages of the streams going up (e.g., the feed stream that is being cooled) should be made high enough to overcome the natural tendency for the colder (and therefore denser) fluid to flow downwards.

When the total plant capacity is high enough, two or more such distillation columns in parallel, preferably working at different pressures, can be used so that the reboilers of the prefractionators absorb heat at different temperatures, making the temperature difference in the combined heat exchanger more even, thereby making the process more reversible, meaning less compression is needed for the process.

If methanol absorption is used to further remove CO₂ from the overhead vapor of the prefractoionator, and for whatever reason significant amounts of pentanes and heavier hydrocarbons enter the methanol loop in the absorption unit, such a contaminated solvent can be replaced by fresh methanol and sold or used as a gasoline blending component.

Heavier hydrocarbon components whose boiling points are greater than that of methanol may accumulate and eventually become a major component of the absorbent, which can reduce the efficiency of the absorption process. However, in practice, when the concentrations of such high boiling components in the feed to the absorption system are extremely low, it will take a long time before a significant effect can be produced. When such a significant effect is observed, replacing the methanol absorbent can be an effective solution.

This invention is illustrated by the following examples, which do not limit the scope thereof. It is readily appreciated by those of ordinary skill in the art that, in view of the present disclosure, modifications can be made to the exemplary process/system and other processes/systems are possible to carry out the present invention.

### Example-1

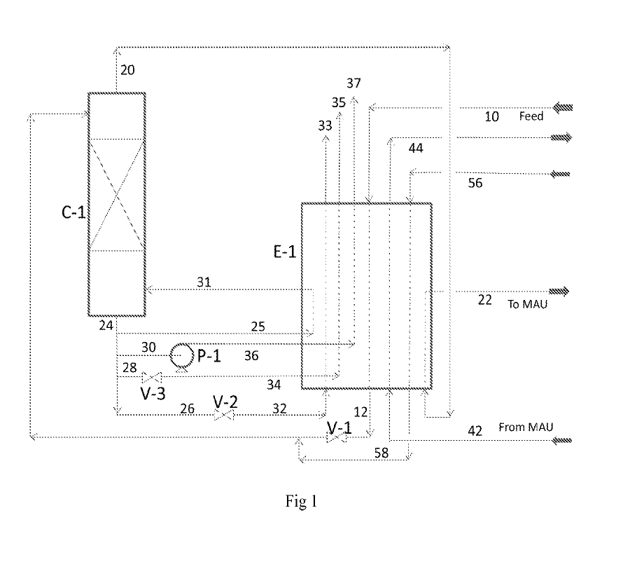

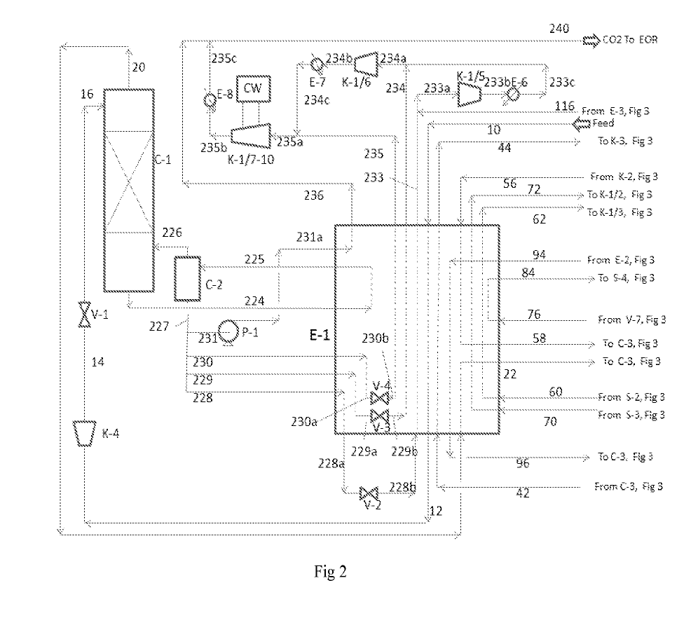

The Process Flow Diagram (PFD) is shown in FIG. 1. In the process, the feed gas mixture of CO₂, methane, and small amounts of nitrogen and heavier hydrocarbons such as ethane, propane, butanes, pentanes, and hexanes at 800 psia and 100° F., in line 10, is first cooled and condensed in the combined reboiler and “second heat exchanger” (to be called Main Heat Exchanger), E-1. The condensed feed stream, in line 12, is let down in pressure to close to 483 psia by the Feed JT-valve, V-1, and fed to the top of the Prefractionator, C-1. The methane enriched overhead vapor, in line 20, from the top of Prefractionator C-1 is heated in the Main Heat Exchanger, E-1, and then taken out of E-1 at an intermediate location and sent for further CO₂ removal in methanol absorption unit (to be called MAU, not shown).

The essentially CO₂ and heavier hydrocarbons (0.1 mol % methane) bottoms liquid, to be called first stream, in line 24, of the Prefractionator, C-1, is split into four substreams. The first stream, in line 25, from the bottom of the Prefaractionator C-1, is fed to an intermediate location of E-1 and at least partially vaporized in E-1. The resultant at least partially vaporized light components depleted substream, in line 31, is sent back to the Prefractionator at a position that is below the packed (or trayed) section of the column and above the liquid level in the sump of the column. That is to say, the reboiler of the Prefractionator is located in an intermediate position of the Main Heat Exchanger E-1.

The second light components-depleted liquid substream, in line 26, is let down in pressure in the LP CO₂ JT-valve to about 200 psia, V-2, and the resultant two phase light components depleted stream, in line 32, is sent to the cold end of the Main Heat Exchanger, E-1, and vaporized and further heated in E-1 as it travels towards E-1's warm end.

The third substream, in line 28, is let down in pressure in MP CO₂ JT valve, V3, to about 345 psia. The resultant two phase light components depleted stream, in line 34, is fed to the Main Heat Exchanger E1 at an intermediate location not far from the cold end and vaporized and further heated as it travels towards the warm end of E-1.

The fourth light components-depleted liquid substream, in line 30, is pumped in light components-depleted liquid pump, P-1, to about 585 psia. The resultant pumped light components depleted stream, in line 36, is fed to the Main Heat Exchanger, E-1, at an intermediate location that is about the same location at which the light components-depleted liquid substream is introduced into E-1, heated, vaporized, and further heated as it travels towards the warm end of E-1.

The heated, vaporized, and further heated second, third, and fourth light components-depleted liquid substreams are taken out of the Main Heat Exchanger E-1, in lines 33, 35, and 37, and can be further compressed in various stages of a CO2 compressor and produced as a high pressure light components depleted product or products, which can be used for enhanced oil recovery in an oilfield, and/or used in other applications.

The essentially methane (and nitrogen) vapor from the top of the methanol absorption column of the MAU (not shown), in line 42, is introduced into the cold end of the Main Heat Exchanger E-1, heated as it travels towards the warm end of E-1 and taken out of E-1 at or near its warm end and is delivered as a fuel gas component, in line 44 (it can be further compressed to a higher pressure if necessary). The vapor stream, in line 56, from a flash tank of the MAU (not shown), after being compressed in a Recycle compressor of the MAU (not shown), is sent to E-1 at or near its warm end for cooling. The E-1 cooled recycle stream, in line 58, is taken out of E-1 from the cold end and sent the top of the prefractionator, e.g., lifted by the condensed feed mixture after its pressure is reduced to the column pressure, likely through an ejector (not shown). That is to say, the at least partially condensed feed mixture from the cold end of the Main Heat Exchanger E-1 is the motive of the ejector. Such an ejector may be able to reduce the discharge pressure of the recycle compressor (not shown), thereby saves some energy for compression of the recycle gas.

To make the process even more efficient, one can use the overhead vapor from the prefractionator, in line 20, and that from the methanol absorption column, in line 42, to cool the methanol before it is further cooled and fed to the top of the methanol absorption column before these two overhead vapor streams are sent to the Main Heat Exchanger. This is not shown FIG. 1.

### Example-2

This process is developed for CO₂ capture from a natural gas containing 65 mol % CO₂, similar to that in the example used by Ross and Cueller. The feed gas is at 1125 psia. The CO₂ product is delivered at 1900 psia, and the (essentially methane) sales gas is delivered 1175 psia. Both are the same as those in Ross and Cueller's paper. Ross and Cueller claimed that using the Ortloff DRCF-SELEXOL hybrid process, they were able to reduce the power consumption from 81.9 MW needed for the conventional SELEXOL-only process to 45.5 MW and capital cost by $50 MM for a 400 MMSCFD feed gas flow plant.

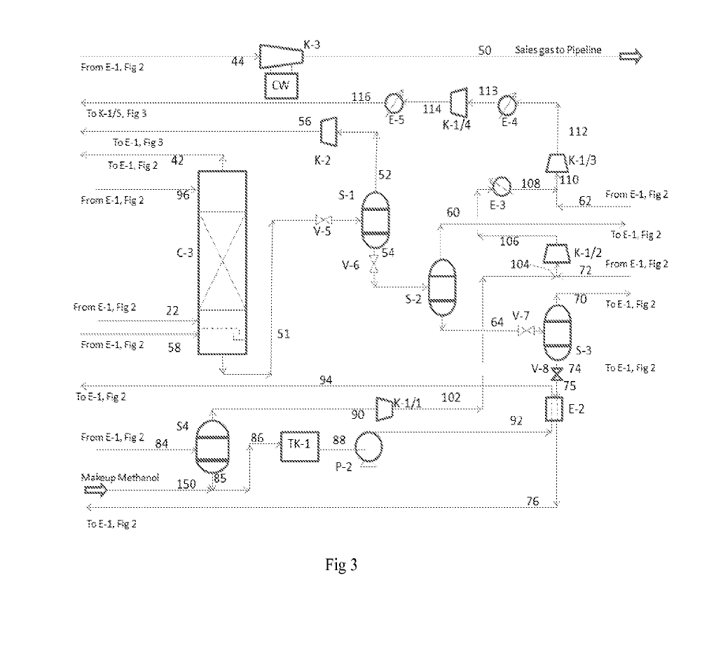

Ross and Cueller did not specify the exact composition of the natural gas, but mentioned “65% CO₂ and 35% hydrocarbon, predominately methane”. We assumed the feed contains 65 mol % CO₂, 32 mol % methane, 1.46 mol % ethane, 0.41 mol % propane, 0.09 mol % isobutene, 0.16 mol % n-butane, 0.09 mol % isopentane, 0.05% n-pentane, 0.24 mol % n-hexane, and 0.5% nitrogen to mimic a “normal” gas composition. FIGS. 2 and 3 show the Process Flow Diagram (PFD) incorporating embodiments of this invention with a methanol absorption process.

In this process, the feed gas mixture, in line 10, is cooled in the combined heat exchanger (to be called Main Heat Exchanger), E-1, and taken out of E-1 at an intermediately location. The resultant cooled and essentially completely “condensed” feed stream (for a feed gas at a pressure higher than its critical pressure, it means at least 5° F. below its critical temperature), in line 12, is expanded in expander, K-4, to generate work and refrigeration, and then further let down in pressure in V-1. The resultant two phase feed stream, in line 16, is fed to the top of the prefractionator, C-1, where it is separated into the light components-depleted bottoms liquid, in line 224, and a CO₂ (and heavier hydrocarbons)-depleted overhead vapor, in line 20.

The other differences between FIG. 2 and FIG. 1 are indicated by the streams in lines starting with 200 and described below: the bottoms light components-depleted liquid from the prefractionator C-1, in line 224, is fed to an intermediate location of the Main Heat Exchanger E-1 in which it is at least partially vaporized. The at least partially vaporized light components-depleted stream, in line 225, is fed into the phase separator, C-2. The vapor stream, in line 226, from the phase separator is sent to the prefractionator at a position below the packed section (or trayed section) of the prefractionator. The liquid from the phase separator C-2, in line 227, is the light components depleted liquid product stream which is essentially free of methane (it is also called “CO₂ stream” in the text below although it also contains most of the heavy hydrocarbons in the feed as well and may still contain a small amount of methane). Note whether to use a phase separator as in this process or not using a phase separator is not dependent on the feed pressure.

The light components depleted product stream, in line 227, is split into four substreams. The first substream, in line 228, is first further cooled in the Main Heat Exchanger, E-1, and the subcooled liquid, in line 228a, from the cold end of E-1 is let down in pressure in the LP CO₂ JT-valve, V-2, to 155 psia. The resultant two phase light components depleted stream, in line 228b, is sent to the cold end of the Main Heat Exchanger, E-1, vaporized and further heated in E-1 as it travels towards E-1's warm end.

The second substream, in line 229, is also further cooled in the Main Heat Exchanger, E-1, and the subcooled liquid, in line 229a, taken out of E-1 at a location not very far from the cold end of E-1, and let down in pressure in the MP CO₂ JT-valve, V-3, to 220 psia. The resultant two phase light components depleted stream, in line 229b, is sent to the Main Heat Exchanger, E-1, at a location that is close to where stream 229a is taken out, vaporized and further heated in E-1 as it travels towards E-1's warm end.

The third substream, in line 230, is also further cooled in the Main Heat Exchanger, E-1. The subcooled liquid, in line 230a, is taken out of E-1 at a location that is further away from the cold end of E-1 than where 229a is taken out, and let down in pressure in the HP CO₂ JT-valve, V-4, to 320 psia. The resultant two phase light components depleted stream, in line 230b, is sent to the Main Heat Exchanger, E-1, at a location close to where stream 230a is taken out, vaporized and further heated in E-1 as it travels towards E-1's warm end.

The fourth light components-depleted liquid substream, in line 231, is pumped in CO₂ pump, P-1, to 1915 psia, which is above the critical pressure of CO₂ and higher than the feed gas pressure. The resultant pumped first stream, in line 231a, is fed to the Main Heat Exchanger, E-1, at an intermediate location that is above where the light components-depleted liquid substream from the bottom, stream 224, of the prefractionator, C-1, is introduced into E-1, heated as it travels towards the warm end of E-1. The heated, “vaporized” (for the supercritical stream, there is no clear distinction between “liquid” and “vapor”), and further heated light components-depleted liquid substreams are taken out of warm end of the main heat exchanger E-1. The first three E-1 heated streams, in lines 233, 234, and 235, are further compressed in various stages of a CO₂ compressor and then mixed with stream 236 and sent back to the oil reservoir for enhanced oil recovery.

The overhead vapor, in line 20, of the prefractionator C-1, which still contains more than 20 mol % CO₂, some ethane, but has less than 100 ppm of pentanes and hexanes, is fed to the cold end of E-1, taken out of E-1 at an intermediate location and is then fed to the absorption column C-3 in FIG. 3 above the bottom stage. The overhead vapor, in line 42, of the absorber C-3, which is an essentially methane gas with less than 2 mol % CO₂, is fed to the cold end of E-1 in FIG. 2 and heated to close to ambient temperature. This close-to-ambient temperature gas, in line 44, is further compressed in Sales Gas Compressor K-3 in FIG. 3 to the desired delivery pressure of 1175 psia (in line 50) and sent to the sales gas pipeline.

The bottoms liquid, in line 51, of the absorber C-3 is let down in pressure, in V-5 (to 190 psia). The resultant two phase stream is separated into a vapor stream, in line 52, and liquid stream, in line 54, in Phase Separator S-1. The vapor stream, in line 52, which contains most of the methane dissolved in bottoms liquid of the absorber along with some CO₂ and other components, is compressed in the recycle compressor, K-2, and resultant higher pressure and higher temperature recycle stream, in line 56, is fed to the warm end of E-1 in FIG. 2 to be cooled. It is then taken out of E-1 at an intermediate location and the resultant cooled recycle gas is fed to the bottom of the absorber, below the bottom stage (likely a tray).

The liquid stream, in line 54, from S-1, is further reduced in pressure in valve V-6 (to 50 psia). The resultant two phase stream is phase-separated in Phase Separator S-2. The vapor, in line 60, from this phase separator is sent to an intermediate position of the Main Heat Exchanger E-1 in FIG. 2 and then taken out of E-1 at its warm end before this essentially CO₂ stream, in line 62, is fed to the 3rd stage of the CO₂ compressor, K-1/3, in FIG. 3. The liquid stream, in line 64, from S-2, is further reduced in pressure, in V-7, to 25 psia. The resultant two phase stream is sent to Phase Separator S-3. The vapor stream, in line 70, is fed to an intermediate location of E-1 in FIG. 2 and heated to close to ambient temperature there. The resultant heated vapor, in line 72, from the warm end of E-1 is combined with the gas stream in line 102, from K-1/1 in FIG. 3. The resultant CO₂ mixture, in line 104, is further compressed in the second stage of the CO₂ compressor K-1/2. The K-1/2 compressed stream, in line 106, is cooled in intercooler, E-3.

The liquid stream, in line 74, from Phase Separator S-3, is let down in pressure in valve V-8. The resultant stream, in line 75, is led to the Economizer, E-2, and heated in E2. The resultant two phase stream from E-2, in line 76, is further heated in E-1 (FIG. 2) at an intermediate location. The E-1 heated two phase stream, in line 84, is taken out of E-1 at an intermediate position and sent to Phase Separator S-4 in FIG. 3. The vapor stream, in line 90, from S-4 is compressed by the first stage of CO₂ compressor K-1/1. The liquid stream, in line 85, from S-4 is fed to the methanol tank, TK-1, where Makeup Methanol, in line 150, is also fed. The methanol from TK-1, in line 92, is pumped by Methanol Pump, P-2. The pumped methanol, in line 92, is first cooled in Economizer E-2. Then this partially cooled methanol, in line 94, is further introduced to an intermediated location of E-1 in FIG. 2 and cooled in the cold section of E-1. The E-1 cooled methanol, in line 96, is fed to the top of the absorber, C-3, in FIG. 3, as the absorbent.

The CO₂, in line 108, corning out of the aftercooler to K-1/2, E-3, is mixed with stream 62 returning from the warm end of E-1 in FIG. 2 and the combined stream, in line 110, is further compressed by K-1/3, cooled by intercooler E-4, and further compressed by K-1/4 (with intercoolers E-5). The CO₂, in line 116, thus obtained is then mixed with the CO₂, in line 233 (in FIG. 2), from the warm end of E-1, further compressed by K-1/5, intercooled in E-6, and further combined with another E-1 heated CO₂ stream, in line 234. The combined CO₂ stream is then further compressed in K-1/6, cooled in E-7. The CO₂ from E-7 further combines with another E-1 heated CO₂ stream, in line 235, and the combined stream is further compressed in the 4 stages of CO₂ compressor (with intercoolers), K-1/7-10. The resultant CO₂, in line 235c, combines with the pumped and E-1 heated CO₂, in line 236, to form the 1900 psia product CO₂ stream, in line 240.

The stream conditions from simulation are shown in Table 1 for 100 lbmol/hr of feed. Note we assumed the temperature of the streams from coolers is 78° F., as is the feed gas. The machinery efficiency numbers used in the simulation are as follows: 85% polytropic efficiency and 96% mechanical efficiency for compressors; 75% pump efficiency and 96% mechanical efficiency for pumps; 75% isentropic efficiency and 94% mechanical efficiency for the expander. Peng-Robinson thermodynamic model was used in the simulation. Both the prefractionator and the absorber in this example have 15 stages. The heat leak was assumed to be 10 Btu/lbmol, leaked into the bottom of the absorber.

The temperature, pressure, flow rate, and mole fractions of the stream obtained from the simulation are listed in Table 1. Table 2 shows a list of power consumed by the compressors and pumps and the power generated by the expander in this process at 100 lbmol/hr feed rate. From this table, we calculated that the power needed for these machines for a 400 MMSCFD plant is 21.8 MW. Assuming we need additional 0.7 MW for pumping the cooling water and other purposes, the total power needed for this process is 22.5 MW, which is only 49.5% of the 45.5 MW using Ortloff's DRCF-SELEXOL hybrid process, or 27.5% of the 81.9 MW using the conventional SELEXOL process.

It should be noted that the pressures of the products, especially that of CO₂, are higher than the feed pressure in this example. This means if we bring the product pressures to that of the feed pressure, so that the true power consumption of the process reflects the power needed for separation, the percent power reduction using the process in FIGS. 2 and 3 is even more pronounced.

Since the cost of the power consuming equipment: the compressors and pumps, are the major cost items in this process, and we expect the equipment in the methanol absorption process, including the absorption column, the phase separators, and the pipelines, to be significantly smaller than those in the SELEXOL process used by the Ortloff DRCF-SELEXOL hybrid process, we expect a great capital cost reduction using this process from that of the Ortloff DRCF-SELEXOL hybrid process as well.

TABLE 1

| | Stream No | | | | | | | |
|---|---|---|---|---|---|---|---|---|
| Mole Frac | 10 | 12 | 14 | 20 | 22 | 42 | 44 | 50 |
| N2 | 0.005 | 0.005 | 0.005 | 0.011286 | 0.011286 | 0.015089 | 0.015089 | 0.015089 |
| methane | 0.32 | 0.32 | 0.32 | 0.721085 | 0.721085 | 0.95259 | 0.95259 | 0.95259 |
| Ethane | 0.0146 | 0.0146 | 0.0146 | 0.012498 | 0.012493 | 0.012467 | 0.012467 | 0.012467 |
| Propane | 0.0041 | 0.0041 | 0.0041 | 0.001113 | 0.001113 | 0.0002 | 0.0002 | 0.0002 |
| Isobutane | 0.0009 | 0.0009 | 0.0009 | 0.000109 | 0.000109 | 2.19E−05 | 2.19E−05 | 2.19E−05 |
| N-butane | 0.0016 | 0.0016 | 0.0016 | 0.000146 | 0.000146 | 3.38E−05 | 3.38E−05 | 3.38E−05 |
| Isopentane | 0.0009 | 0.0009 | 0.0009 | 3.11E−05 | 3.11E−05 | 9.54E−06 | 9.54E−06 | 9.54E−06 |
| N-pentane | 0.0005 | 0.0005 | 0.0005 | 1.22E−05 | 1.22E−05 | 3.81E−06 | 3.81E−06 | 3.81E−06 |
| Hexane | 0.0024 | 0.0024 | 0.0024 | 1.52E−05 | 1.52E−05 | 5.90E−05 | 5.90E−05 | 5.90E−05 |
| CO2 | 0.65 | 0.65 | 0.65 | 0.253709 | 0.253709 | 0.019466 | 0.019466 | 0.019466 |
| Methanol | 0 | 0 | 0 | 0 | 0 | 0.000114 | 0.000114 | 0.000114 |
| Total Flow lbmol/hr | 100 | 100 | 100 | 44.30286 | 44.30288 | 33.07322 | 33.07322 | 33.07322 |
| Total Flow lb/hr | 3495.265 | 3495.265 | 3495.265 | 1040.851 | 1040.851 | 560.7178 | 560.7178 | 560.7178 |
| Total Flow ouft/hr | 328.9749 | 80.27614 | 118.6071 | 317.5273 | 396.9713 | 281.7132 | 390.6073 | 137.9809 |
| Temperature F. | 77.91114 | −30 | −41.9817 | −59.4695 | −10 | −39.7028 | 68.54722 | 78 |
| Pressure psia | 1124 | 1119 | 729.1887 | 455.5744 | 453.5744 | 448.2264 | 446.2264 | 1173 |
| Vapor Frac | 1 | 0 | 0.138378 | 1 | 1 | 1 | 1 | 1 |
| Liquid Frac | 0 | 1 | 0.861622 | 0 | 0 | 0 | 0 | 0 |
| | Stream No | | | | | | | |
| Mole Frac | 51 | 52 | 54 | 56 | 58 | 60 | 62 | 64 |
| N2 | 7.75E−05 | 0.002442 | 1.18E−09 | 0.002442 | 0.002442 | 0.000164 | 0.000164 | 1.83E−07 |
| methane | 0.018272 | 0.482895 | 0.005368 | 0.482895 | 0.482895 | 0.073117 | 0.073117 | 0.000195 |
| Ethane | 0.002275 | 0.021998 | 0.001723 | 0.021995 | 0.021995 | 0.018934 | 0.018934 | 0.000414 |
| Propane | 0.000614 | 0.001811 | 0.000581 | 0.001811 | 0.001811 | 0.003585 | 0.003585 | 0.000351 |
| Isobutane | 6.03E−05 | 0.000147 | 5.79E−05 | 0.000147 | 0.000147 | 0.000322 | 0.000322 | 3.77E−05 |
| N-butane | 9.98E−05 | 0.000116 | 9.94E−05 | 0.000116 | 0.000116 | 0.000302 | 0.000302 | 8.39E−05 |
| Isopentane | 2.28E−05 | 1.98E−05 | 2.29E−05 | 1.98E−05 | 1.98E−05 | 5.54E−05 | 5.54E−05 | 2.04E−04 |
| N-pentane | 1.12E−05 | 6.53E−05 | 1.45E−05 | 6.53E−06 | 6.53E−06 | 1.88E−05 | 1.88E−05 | 1.08E−05 |
| Hexane | 1.84E−05 | 6.29E−05 | 1.88E−05 | 6.29E−05 | 6.29E−05 | 1.92E−05 | 1.92E−05 | 1.87E−05 |
| CO2 | 0.149499 | 0.489875 | 0.140046 | 0.489875 | 0.489875 | 0.902389 | 0.902389 | 0.081834 |
| Methanol | 0.82905 | 0.000684 | 0.852056 | 0.000684 | 0.000684 | 0.001094 | 0.001094 | 0.917034 |
| Total Flow lbmol/hr | 84.43105 | 2.281565 | 82.14949 | 2.281566 | 2.281566 | 5.827823 | 5.827823 | 76.32166 |
| Total Flow lb/hr | 2832.481 | 68.80151 | 2763.68 | 68.80151 | 68.80151 | 243.0004 | 243.0004 | 2520.679 |
| Total Flow ouft/hr | 52.83283 | 53.64387 | 51.13117 | 28.59973 | 19.82495 | 531.428 | 606.7352 | 46.81745 |
| Temperature F. | −5.1127 | −6.67298 | −6.67296 | 116.9532 | −5 | −20.3989 | 32 | −20.3989 |
| Pressure psia | 448.4364 | 190 | 190 | 450.5 | 448.5 | 50 | 49.5 | 50 |
| Vapor Frac | 0 | 1 | 0 | 1 | 0.999596 | 1 | 1 | 0 |
| Liquid Frac | 1 | 0 | 1 | 0 | 0.000401 | 0 | 0 | 1 |
| | Stream No | | | | | | | |
| Mole Frac | 70 | 72 | 74 | 75 | 76 | 84 | 85 | 86 |
| N2 | 4.97E−05 | 4.97E−06 | 2.58E−09 | 2.58E−09 | 2.58E−09 | 2.58E−09 | 2.31E−11 | 2.30E−11 |
| methane | 0.005197 | 0.005197 | 6.47E−08 | 6.47E−08 | 6.47E−08 | 6.47E−08 | 1.31E−07 | 1.31E−07 |
| Ethane | 0.008859 | 0.008859 | 9.61E−05 | 9.61E−05 | 9.61E−05 | 9.61E−05 | 1.12E−05 | 1.11E−05 |
| Propane | 0.004153 | 0.004153 | 0.000208 | 0.000208 | 0.000208 | 0.000208 | 7.05E−05 | 7.04E−05 |
| Isobutane | 0.000409 | 0.000409 | 2.38E−05 | 2.38E−05 | 2.38E−05 | 2.38E−05 | 9.27E−06 | 9.26E−05 |
| N-butane | 0.00048 | 0.00048 | 6.90E−05 | 6.90E−05 | 6.90E−05 | 6.90E−05 | 3.99E−05 | 3.99E−05 |
| Isopentane | 9.52E−05 | 9.52E−05 | 1.76E−05 | 1.76E−05 | 1.76E−05 | 1.76E−05 | 1.15E−05 | 1.15E−05 |
| N-pentane | 3.39E−05 | 3.39E−05 | 9.93E−08 | 9.93E−08 | 9.93E−08 | 9.93E−08 | 7.83E−06 | 7.32E−05 |
| Hexane | 3.73E−05 | 3.73E−05 | 1.80E−05 | 1.80E−05 | 1.80E−05 | 1.80E−05 | 1.50E−05 | 1.50E−05 |
| CO2 | 0.979281 | 0.979281 | 0.048059 | 0.048059 | 0.048059 | 0.048059 | 0.012829 | 0.012805 |
| Methanol | 0.001449 | 0.001449 | 0.951492 | 0.951492 | 0.951492 | 0.951492 | 0.987005 | 0.987028 |
| Total Flow lbmol/hr | 2.768186 | 2.768186 | 73.55348 | 73.55348 | 73.55348 | 73.55348 | 70.81183 | 70.91985 |
| Total Flow lb/hr | 121.0829 | 121.0829 | 2399.596 | 2399.596 | 2399.596 | 2399.596 | 2280.085 | 2283.545 |
| Total Flow ouft/hr | 503.5197 | 588.9182 | 44.80562 | 333.112 | 866.3712 | 1001.704 | 45.03428 | 45.10495 |
| Temperature F. | −28.1233 | 32 | −28.1233 | −31.2589 | 24.50257 | 32 | 32 | 32.0585 |
| Pressure psia | 25 | 24.5 | 25 | 17 | 16 | 15 | 15 | 15 |
| Vapor Frac | 1 | 1 | 0 | 0.014718 | 0.034695 | 0.037274 | 0 | 0 |
| Liquid Frac | 0 | 0 | 1 | 0.985282 | 0.965306 | 0.962776 | 1 | 1 |
| | Stream No | | | | | | | |
| Mole Frac | 90 | 92 | 94 | 96 | 102 | 104 | 106 | 108 |
| N2 | 6.88E−08 | 2.30E−11 | 2.30E−11 | 2.30E−11 | 6.88E−08 | 2.53E−06 | 2.53E−06 | 2.53E−06 |
| methane | 0.00017 | 1.31E−07 | 1.31E−07 | 1.31E−07 | 0.0017 | 0.002696 | 0.002696 | 0.002696 |
| Ethane | 0.002289 | 1.11E−05 | 1.11E−05 | 1.11E−05 | 0.002289 | 0.00559 | 0.00559 | 0.00559 |
| Propane | 0.003765 | 7.04E−05 | 7.04E−05 | 7.04E−05 | 0.003765 | 0.00396 | 0.00396 | 0.00396 |
| Isobutane | 0.000398 | 9.26E−06 | 9.26E−06 | 9.26E−06 | 0.000398 | 0.000404 | 0.000404 | 0.000404 |
| N-butane | 0.000819 | 3.99E−05 | 3.99E−05 | 3.99E−05 | 0.000819 | 0.000649 | 0.000649 | 0.000649 |
| Isopentane | 0.000174 | 1.15E−05 | 1.15E−05 | 1.15E−05 | 0.000174 | 0.000134 | 0.000134 | 0.000134 |
| N-pentane | 7.70E−05 | 7.32E−06 | 7.32E−06 | 7.32E−06 | 7.70E−05 | 5.53E−05 | 5.53E−05 | 5.53E−05 |
| Hexane | 9.57E−05 | 1.50E−05 | 1.50E−05 | 1.50E−05 | 9.57E−05 | 6.63E−05 | 6.63E−05 | 6.63E−05 |
| CO2 | 0.957978 | 0.012809 | 0.012809 | 0.012809 | 0.957978 | 0.968681 | 0.968681 | 0.968681 |
| Methanol | 0.034234 | 0.987026 | 0.987026 | 0.987026 | 0.034234 | 0.017762 | 0.017762 | 0.017762 |
| Total Flow lbmol/hr | 2.741642 | 70.91985 | 70.91985 | 70.91985 | 2.741642 | 5.509828 | 5.509828 | 5.509828 |
| Total Flow lb/hr | 119.5116 | 2283.545 | 2283.545 | 2283.545 | 119.5116 | 240.5945 | 240.5945 | 240.5945 |
| Total Flow ouft/hr | 956.67 | 45.14895 | 43.14981 | 42.7897 | 655.65 | 1255.767 | 720.4072 | 629.6725 |
| Temperature F. | 32 | 33.31718 | −28 | −40 | 99.59738 | 66.0909 | 175.3859 | 78 |
| Pressure psia | 15 | 514.3582 | 513.3582 | 512.8582 | 24.5 | 24.5 | 51.5 | 49.5 |
| Vapor Frac | 1 | 0 | 0 | 0 | 1 | 1 | 1 | 1 |
| Liquid Frac | 0 | 1 | 1 | 1 | 0 | 0 | 0 | 0 |
| | Stream No | | | | | | | |
| Mole Frac | 110 | 112 | 113 | 114 | 116 | 150 | 227 | 228 |
| N2 | 8.53E−05 | 8.53E−05 | 8.53E−05 | 8.53E−05 | 8.53E−05 | 0 | 1.21E−10 | 1.21E−10 |
| methane | 0.038894 | 0.038894 | 0.038894 | 0.038894 | 0.038894 | 0 | 0.000966 | 0.000966 |
| Ethane | 0.012449 | 0.012449 | 0.012449 | 0.012449 | 0.012449 | 0 | 0.016276 | 0.016276 |
| Propane | 0.003767 | 0.003767 | 0.003767 | 0.003767 | 0.003767 | 0 | 0.006476 | 0.006476 |
| Isobutane | 0.000361 | 0.000361 | 0.000361 | 0.000361 | 0.000361 | 0 | 0.001529 | 0.001529 |
| N-butane | 0.000471 | 0.000471 | 0.000471 | 0.000471 | 0.000471 | 0 | 0.002757 | 0.002757 |
| Isopentane | 9.38E−05 | 9.38E−05 | 9.38E−05 | 9.38E−05 | 9.38E−05 | 0 | 0.001591 | 0.001591 |
| N-pentane | 3.66E−05 | 3.66E−05 | 3.66E−05 | 3.66E−05 | 3.66E−05 | 0 | 0.000883 | 0.000883 |
| Hexane | 4.21E−05 | 4.21E−05 | 4.21E−05 | 4.21E−05 | 4.21E−05 | 0 | 0.004297 | 0.004297 |
| CO2 | 0.934606 | 0.934606 | 0.934606 | 0.934606 | 0.934606 | 0 | 0.96522 | 0.96522 |
| Methanol | 0.009194 | 0.009194 | 0.009194 | 0.009194 | 0.009194 | 1 | 0 | 0 |
| Total Flow lbmol/hr | 11.33765 | 11.33765 | 11.33765 | 11.33765 | 11.33765 | 0.108011 | 55.69714 | 5.569714 |
| Total Flow lb/hr | 483.5949 | 483.5949 | 483.5949 | 483.5949 | 483.5949 | 3.460882 | 2454.413 | 245.4413 |
| Total Flow ouft/hr | 1236.958 | 865.7162 | 803.3293 | 484.7478 | 402.462 | 0.070732 | 42.24707 | 4.224707 |
| Temperature F. | 54.5584 | 123.2048 | 78 | 179.3042 | 78 | 70 | 24.84214 | 24.84214 |
| Pressure psis | 49.5 | 80 | 79 | 155 | 153 | 17 | 455.7844 | 455.7844 |
| Vapor Frac | 1 | 1 | 1 | 1 | 1 | 0 | 0 | 0 |
| Liquid Frac | 0 | 0 | 0 | 0 | 0 | 1 | 1 | 1 |
| | Stream No | | | | | | | |
| Mole Frac | 228a | 228b | 229 | 229a | 229b | 230 | 230a | 230b |
| N2 | 1.21E−10 | 1.21E−10 | 1.21E−10 | 1.21E−10 | 1.21E−10 | 1.21E−10 | 1.21E−10 | 1.21E−10 |
| methane | 0.000966 | 0.000966 | 0.000966 | 0.000966 | 0.000966 | 0.000966 | 0.000966 | 0.000966 |
| Ethane | 0.016276 | 0.016276 | 0.016276 | 0.016276 | 0.016276 | 0.016276 | 0.016276 | 0.016276 |
| Propane | 0.006476 | 0.006476 | 0.006476 | 0.000476 | 0.006476 | 0.006476 | 0.006476 | 0.006476 |
| Isobutane | 0.001529 | 0.001529 | 0.001529 | 0.001529 | 0.001529 | 0.001529 | 0.001529 | 0.001529 |
| N-butane | 0.002757 | 0.002757 | 0.002757 | 0.002757 | 0.002757 | 0.002757 | 0.002757 | 0.002757 |
| Isopentane | 0.001591 | 0.001591 | 0.001591 | 0.001591 | 0.001591 | 0.001591 | 0.001591 | 0.001591 |
| N-pentane | 0.000883 | 0.000883 | 0.000883 | 0.000883 | 0.000883 | 0.000883 | 0.000883 | 0.000883 |
| Hexane | 0.004297 | 0.004297 | 0.004297 | 0.004297 | 0.004297 | 0.004297 | 0.004297 | 0.004297 |
| CO2 | 0.96522 | 0.96522 | 0.96522 | 0.96522 | 0.96522 | 0.96522 | 0.96522 | 0.96522 |
| Methanol | 0 | 0 | 0 | 0 | 0 | 0 | | |
| Total Flow lbmol/hr | 5.569714 | 5.569714 | 11.6964 | 11.6964 | 11.6964 | 20.38515 | 20.38515 | 20.38515 |
| Total Flow lb/hr | 245.4411 | 245.4413 | 515.4263 | 515.4263 | 515.4263 | 898.3153 | 898.3153 | 898.3153 |
| Total Flow ouft/hr | 3.644843 | 3.64516 | 8.871884 | 7.95683 | 7.951795 | 15.46243 | 13.92509 | 13.91954 |
| Temperature F. | −40 | −39.9526 | 24.84214 | −20 | −20.3083 | 24.84214 | −18 | −18.1916 |
| Pressure psis | 454.7844 | 155 | 455.7844 | 453.7844 | 220 | 455.7844 | 454.7844 | 320 |
| Vapor Frac | 0 | 0 | 0 | 0 | 0 | 0 | 0 | 0 |
| Liquid Frac | 1 | 1 | 1 | 1 | 1 | 1 | 1 | 1 |
| | Stream No | | | | | | | |
| Mole Frac | 110 | 112 | 113 | 114 | 116 | 150 | 227 | 228 |
| N2 | 8.53E−05 | 8.53E−05 | 8.53E−05 | 8.53E−05 | 8.53E−05 | 0 | 1.21E−10 | 1.21E−10 |
| methane | 0.038894 | 0.038894 | 0.038894 | 0.038894 | 0.038894 | 0 | 0.000966 | 0.000966 |
| Ethane | 0.012449 | 0.012449 | 0.012449 | 0.012449 | 0.012449 | 0 | 0.016276 | 0.016276 |
| Propane | 0.003767 | 0.003767 | 0.003767 | 0.003767 | 0.003767 | 0 | 0.006476 | 0.006476 |
| Isobutane | 0.000361 | 0.000361 | 0.000361 | 0.000361 | 0.000361 | 0 | 0.001529 | 0.001529 |
| N-butane | 0.000471 | 0.000471 | 0.000471 | 0.000471 | 0.000471 | 0 | 0.002757 | 0.002757 |
| Isopentane | 9.38E−05 | 9.38E−05 | 9.38E−05 | 9.38E−05 | 9.38E−05 | 0 | 0.001591 | 0.001591 |
| N-pentane | 3.66E−05 | 3.66E−05 | 3.66E−05 | 3.66E−05 | 3.66E−05 | 0 | 0.000883 | 0.000883 |
| Hexane | 4.21E−05 | 4.21E−05 | 4.21E−05 | 4.21E−05 | 4.21E−05 | 0 | 0.004297 | 0.004297 |
| CO2 | 0.934606 | 0.934606 | 0.934606 | 0.934606 | 0.934606 | 0 | 0.96522 | 0.96522 |
| Methanol | 0.009194 | 0.009194 | 0.009194 | 0.009194 | 0.009194 | 1 | 0 | 0 |
| Total Flow lbmol/hr | 11.33765 | 11.33765 | 11.33765 | 11.33765 | 11.33765 | 0.108011 | 55.69714 | 5.569714 |
| Total Flow lb/hr | 483.5949 | 483.5949 | 483.5949 | 483.5949 | 483.5949 | 3.460882 | 2454.413 | 245.4413 |
| Total Flow ouft/hr | 1236.958 | 865.7162 | 803.3293 | 484.7478 | 402.462 | 0.070732 | 42.24707 | 4.224707 |
| Temperature F. | 54.5584 | 123.2048 | 78 | 179.3042 | 78 | 70 | 24.84214 | 24.84214 |
| Pressure psis | 49.5 | 80 | 79 | 155 | 153 | 17 | 455.7844 | 455.7844 |
| Vapor Frac | 1 | 1 | 1 | 1 | 1 | 0 | 0 | 0 |
| Liquid Frac | 0 | 0 | 0 | 0 | 0 | 1 | 1 | 1 |
| | Stream No | | | | | | | |
| Mole Frac | 228a | 228b | 229 | 229a | 229b | 230 | 230a | 230b |
| N2 | 1.21E−10 | 1.21E−10 | 1.21E−10 | 1.21E−10 | 1.21E−10 | 1.21E−10 | 1.21E−10 | 1.21E−10 |
| methane | 0.000966 | 0.000966 | 0.000966 | 0.000966 | 0.000966 | 0.000966 | 0.000966 | 0.000966 |
| Ethane | 0.016276 | 0.016276 | 0.016276 | 0.016276 | 0.016276 | 0.016276 | 0.016276 | 0.016276 |
| Propane | 0.006476 | 0.006476 | 0.006476 | 0.000476 | 0.006476 | 0.006476 | 0.006476 | 0.006476 |
| Isobutane | 0.001529 | 0.001529 | 0.001529 | 0.001529 | 0.001529 | 0.001529 | 0.001529 | 0.001529 |
| N-butane | 0.002757 | 0.002757 | 0.002757 | 0.002757 | 0.002757 | 0.002757 | 0.002757 | 0.002757 |
| Isopentane | 0.001591 | 0.001591 | 0.001591 | 0.001591 | 0.001591 | 0.001591 | 0.001591 | 0.001591 |
| N-pentane | 0.000883 | 0.000883 | 0.000883 | 0.000883 | 0.000883 | 0.000883 | 0.000883 | 0.000883 |
| Hexane | 0.004297 | 0.004297 | 0.004297 | 0.004297 | 0.004297 | 0.004297 | 0.004297 | 0.004297 |
| CO2 | 0.96522 | 0.96522 | 0.96522 | 0.96522 | 0.96522 | 0.96522 | 0.96522 | 0.96522 |
| Methanol | 0 | 0 | 0 | 0 | 0 | 0 | | |
| Total Flow lbmol/hr | 5.569714 | 5.569714 | 11.6964 | 11.6964 | 11.6964 | 20.38515 | 20.38515 | 20.38515 |
| Total Flow lb/hr | 245.4411 | 245.4413 | 515.4263 | 515.4263 | 515.4263 | 898.3153 | 898.3153 | 898.3153 |
| Total Flow ouft/hr | 3.644843 | 3.64516 | 8.871884 | 7.95683 | 7.951795 | 15.46243 | 13.92509 | 13.91954 |
| Temperature F. | −40 | −39.9526 | 24.84214 | −20 | −20.3083 | 24.84214 | −18 | −18.1916 |
| Pressure psis | 454.7844 | 155 | 455.7844 | 453.7844 | 220 | 455.7844 | 454.7844 | 320 |
| Vapor Frac | 0 | 0 | 0 | 0 | 0 | 0 | 0 | 0 |
| Liquid Frac | 1 | 1 | 1 | 1 | 1 | 1 | 1 | 1 |

TABLE 2

| Machine | Power (HP) consumed |
|---|---|
| CO₂ compressor stage 1, K-1/1 | 0.77 |
| CO₂ compressor stage 2, K-1/2 | 2.24 |
| CO₂ compressor stage 3, K-1/3 | 2.79 |
| CO₂ compressor stage 4, K-1/4 | 4.16 |
| CO₂ compressor stage 5, K-1/5 | 3.17 |
| CO₂ compressor stage 6, K-1/6 | 5.49 |
| CO₂ compressor stage 7-8, K-1/7-10 | 30.94 |
| Recycle compressor, K-2 | 0.88 |
| Sales gas compressor, K-3 | 16.08 |
| CO₂ pump, P-1 | 4.91 |
| Methanol pump, P-2 | 2.34 |
| Expander, K-4 | −1.68 |
| Net power needed | 71.99 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A process for separating a stream of a feed mixture, wherein the feed mixture comprises greater than 26 mol % CO₂, and 0.1 mol % to 15 mol % in total one or more heavier hydrocarbons, wherein the feed mixture contains at least 0.0001 mol % in total of one or more pentanes and hexanes, comprising:
a) cooling at least a portion of the feed mixture in reboiler of a distillation column, feeding at least a portion of the reboiler-cooled feed mixture to a second heat exchanger to further cool the feed mixture to below 32° F., and sending at least a portion of the feed mixture cooled to below 32° F. to the distillation column, wherein the feed mixture is separated into at least a first stream containing 10 mol % or less light components and a second stream having a mole fraction of the light components higher than the mole fraction of the light components of the feed mixture;
b) producing a second stream from the top or near the top of the distillation column, and sending the second stream to a physical absorption unit comprising an absorbent comprising at least one component selected from the group consisting of methanol, N-Methyl-2-pyrrolidone, propylene carbonate, and sulphinol;
c) introducing the first stream from a position at or near the bottom of the distillation column to the reboiler wherein the first stream is at least partially vaporized, and letting the at least partially vaporized first stream flow out of the reboiler; and
d) routing at least a portion of the vapor of the at least partially vaporized light components depleted stream back to the distillation column and producing a light components-depleted liquid product stream.

2. The process of claim 1, wherein the feed mixture is first cooled in a warmer section of the second heat exchanger before it is fed to the reboiler and then a colder section of the second heat exchanger.

3. The process of claim 1, wherein the reboiler and the second heat exchanger are integrated into a combined heat exchanger.

4. The process of claim 1, wherein the feed mixture cooled to below 32° F. is fed to the distillation column on or above the top stage or packing layer of the distillation column.

5. The process of claim 1, wherein at least a portion of the light components-depleted liquid product stream is heated in the second heat exchanger against cooling of the feed stream.

6. The process of claim 1, wherein at least a portion of the light components-depleted liquid product stream is sent to a second distillation column for further separation.

7. The process of claim 1, wherein the feed mixture cooled to below 32° F. in step a) is expanded in a dense fluid expander before being fed to the distillation column.

8. The process of claim 1, wherein the absorbent of the CO₂ absorption unit in step b) is substantially methanol.

9. The process of claim 1, wherein the feed mixture cooled to below 32° F. is expanded in a dense fluid expander before being fed to the distillation column.

10. A process for separating a stream of a feed mixture, wherein the refrigeration is solely provided by the streams of the process and wherein the feed mixture comprises greater than 26 mol % CO₂, and 0.1 mol % to 15 mol % in total one or more heavier hydrocarbons, wherein the feed mixture contains at least 0.0001 mol % in total one or more of pentanes and hexanes, the process comprises:
a) cooling the feed mixture in the reboiler of a distillation column, feeding the reboiler-cooled feed mixture to a second heat exchanger to further cool the feed mixture to below 32° F., and sending the resultant cooled feed mixture to a distillation column, wherein the feed mixture is separated into at least a first stream containing 10 mol % or less light components and a second stream having a mole fraction of the light components higher than the mole fraction of the light components of the feed mixture;
b) producing the second stream from the top or near the top of the distillation column and sending the second stream to an absorption unit for further separation of CO₂ from methane;
c) introducing a first stream from a position at or near the bottom of the distillation column to the reboiler, wherein the first stream is at least partially vaporized, routing the at least partially vaporized first stream out of the reboiler, and feeding at least a portion of the vapor of the resultant at least partially vaporized light components-depleted stream back to the distillation column;
d) producing a light components-depleted liquid product stream;
e) splitting the light components-depleted liquid product stream from the distillation column into at least two light components-depleted liquid product substreams,
f) letting down the pressure of a light components-depleted liquid product substream and sending it to a position in the colder section of the second heat exchanger wherein the light components-depleted liquid substream is heated, completely vaporized, and further heated as it travels from the colder section to the warmer section of the second heat exchanger, and taking the resultant vapor out of the second heat exchanger at or close to the warm end of the heat exchanger; and
g) boosting the pressure of a second light components-depleted liquid product substream to a level that is greater than that in the distillation column, sending the pressure-boosted second light components-depleted liquid product substream to an intermediate location of the second heat exchanger wherein the second light components-depleted liquid product substream is heated as it travels to the warmer section of the second heat exchanger, and taking the resultant heated stream out of the second heat exchanger at or close to its warm end.

11. The process of claim 10, wherein the feed mixture is first cooled in the warm section of the second heat exchanger before the feed mixture is fed to the reboiler and the cold section of the second heat exchanger.

12. The process of claim 10, wherein the reboiler and the second heat exchanger are integrated into a combined heat exchanger.

13. The process of claim 10, wherein the light components-depleted liquid product substream to be let down in pressure is first cooled to a temperature that is lower than the temperature at the bottom of the distillation column before its pressure is reduced.

14. A system for separating a stream of a gas mixture, wherein the feed mixture comprises greater than 26 mol % CO₂, and 0.1 mol % to 15 mol % in total one or more of heavier hydrocarbons, wherein the feed mixture contains at least 0.0001 mol % in total one or more of pentanes and hexanes, the system comprises a distillation column with a reboiler, a second heat exchanger, and a physical absorption system using an absorbent comprising at least one selected from the group consisting of methanol, N-methyl-2-pyrrolidone, propylene carbonate, and sulphinol, the system further comprising:
a) a feed mixture inlet of the distillation column for receiving the feed mixture, a passage for the feed mixture to be cooled in the reboiler, a passage for cooling the feed mixture in the second heat exchanger, a conduit for connecting the reboiler with the feed mixture passage of the second heat exchanger, a conduit for connecting the feed mixture passage in the second heat exchanger with the feed mixture inlet of the distillation column, a first outlet on the distillation column, located at or near the bottom of the distillation column, for removing a first stream from the distillation column, a second outlet on the distillation column, located at or near the top of the distillation column, for removing said second stream from the distillation column, a conduit for connecting said second outlet on the distillation column with the physical absorption CO₂ removal unit, and
b) a passage in the reboiler for at least partially vaporizing said first stream, a means for connecting the distillation column at a position near its bottom with a first end of said passage of the reboiler, and a means for connecting a second end of said passage at the second end of the reboiler with the distillation column at a position near the bottom of the distillation column for admitting at least a portion of the vapor of the at least partially vaporized first stream from said passage in the reboiler into the distillation column, and an outlet for a liquid components-depleted liquid product stream to leave the distillation column.

15. The system of claim 14, wherein the second heat exchanger has a warmer section and a colder section, both warmer section and colder section having passages for cooling the feed mixture, a conduit for connecting the feed mixture passage in the warmer section of the second heat exchanger with the feed mixture passage in the reboiler, and a conduit for connecting the feed mixture passage in the reboiler with the feed mixture passage in the colder section of the second heat exchanger.

16. The system of claim 14, wherein the second heat exchanger and the reboiler are integrated into a combined heat exchanger.

17. The system of claim 14, wherein the feed mixture inlet of the distillation column is located on or above the top tray or layer of packing of the distillation column.

18. The system of claim 14, further comprising a passage in the second heat exchanger for heating at least a portion of the light components-depleted liquid product stream and a means for connecting said passage for the light components-depleted liquid product stream with the distillation column.

19. The system of claim 14, further comprising a second distillation column for further separation of at least a portion of the light components-depleted liquid product stream from the first distillation column and a means for connecting the first distillation column with the second distillation column.

20. The system of claim 14, wherein the absorbent of the CO₂ absorption unit in b) is substantially methanol.
